(19) United States
(12) Patent Application Publication
(10) Pub. No.: US 2004/0151675 A1
(43) Pub. Date: Aug. 5, 2004

(54) Oral hygiene

(76) Inventor: Grady F. Lawrence, Salisbury, NC (US)

Correspondence Address: Clifton T. Hunt, Jr., #705, 4812 Six Forks Road, Raleigh, NC, 27609, US

(21) Appl. No.: 10/723,764

(22) Filed: Nov. 26, 2003

Related U.S. Application Data
(60) Provisional application No. 60/430,208, filed on Dec. 2, 2002.

(51) Int. Cl. A61K 7/20

(52) U.S. Cl. 424/53

(57) ABSTRACT

An antibacterial solution that cleans the plastic gum portion and enamel tooth portion of dentures without the use effervescence. It is known that effervescence contributes to the deterioration of the plastic portions of dentures, yet many of today's denture cleaners rely on effervescence as the cleaning agent. The antibacterial solution of this invention sanitizes the dentures while cleaning them and, at the same time, preserves the plastic portions of the dentures by avoiding the use of effervescence.

### CROSS REFERENCE TO RELATED APPLICATIONS

This nonprovisional patent application claims the benefit of Provisional Patent Application No. 60/430,208 filed Dec. 2, 2002 for ORAL HYGIENE.

### FIELD OF THE INVENTION

This invention relates to the cleaning and sanitizing of dentures and of the oral cavity (mouth).

### BACKGROUND OF THE INVENTION

The Surgeon General of the United States issued a report on oral health in June, 2001—Oral Health In America: A Report of the Surgeon General (the Report). In announcing his report, the Surgeon General stated:

“The mouth is a mirror of the body, and it is critical to overall health and well-being.”

The report links opportunistic infections with microorganisms commonly found in the mouth. See Chapter 5 in the Report, The Mouth as a Portal of Entry for Infection.

On Nov. 20, 2001, the Surgeon General, Dr. Satcher, convened a meeting of a Partnership Network in Washington, D.C. that involved representatives of nearly 100 organizations. The purpose of the meeting was to initiate action to develop a National Oral Health Plan. Organizations participating in the Partnership Network include the American Medical Association, the Association of State and Territorial Health Officials, the Association of Academic Health Centers, and the American Dental Association. It is the intent of this invention to contribute to national oral health.

### SUMMARY OF THE INVENTION

The object of the invention is to obtain and maintain oral hygiene. For that purpose, there is provided an antibacterial solution that cleans and preserves dentures. This antibacterial cleaning solution is hereinafter called The Solution.

Effervescence is known to cause minor deterioration of the gum portions of dentures each time the dentures are exposed to effervescence. These minor deteriorations of the dentures are cumulative and shorten the useful life of dentures.

Unlike many denture cleaners, The Solution functions effectively without effervescence. The Solution is effectively used to simultaneously sanitize and clean conventional dentures having a plastic gum portion and an enamel tooth portion. The useful life of dentures is increased by cleaning them while sanitizing them with The Solution.

The Solution is also effective to eliminate the transfer of bacteria from toothbrush to mouth.

The Solution fulfills its contribution to oral hygiene by being able to serve as a safe and effective mouthwash.

### DETAILED DESCRIPTION OF THE INVENTION

The Solution of this invention is made from the following formulation:

| | % BY | WEIGHT | AMOUNT |
|---|---|---|---|
| MAIN MIX | WEIGHT | (LBS) | (gals) |
| Water | 90.81% | 60,561.97 | 7,279.44 |
| 50% Hydrogen Peroxide | 9.00% | 6,002.64 | 599.664 |
| Phosphoric Acid | 0.04% | 28.012 | |
| Sodium Lauryl Sulfate | 0.10% | 100.044 | |
| Baking Soda | 0.05% | 100.04 | |
| Flavoring | 0.00% | 0.17 | |
| FD&C blue #1 | 0.00% | 0.16 | |
| FD&C yellow #5 | 0.00% | 0.173 | |

The colors are optional. Any desired color, or no color, may be used as desired. Similarly, the flavoring is optional. The Solution may be made without any flavoring. Or, The Solution may be made with the flavor of choice such as, for example, mint, cinnamon, or vanilla.

The ingredients are mixed at room temperature. The resulting 4.5% hydrogen peroxide becomes the antibacterial agent and the sodium lauryl sulfate and the baking soda become the cleaning agents. The baking soda cleans and whitens the enamel portion of dentures.

The sodium lauryl sulfate in the main mix cleans a denture by removing stain and food particles from the denture while the 4.5% hydrogen peroxide destroys harmful bacteria on the denture. Tests have shown that The Solution kills upon contact 99% of the harmful bacteria on a denture.

The phosphoric acid in the main mix is a reagent that causes the hydrogen peroxide to break up into the free radicals which are bactericidal.

The Solution may be packaged for distribution as desired, but is preferably packaged in 16 oz. bottles for convenient use by the consumer.

No water or effervescence is used during the treatment of dentures with The Solution. The preferred method of treating dentures with The Solution is to provide a small bowl of suitable size to hold the dentures. The dentures are placed in the bowl, and enough of The Solution is poured into the bowl to cover the dentures. The dentures remain covered by The Solution for at least fifteen minutes, preferably overnight, to be simultaneously sanitized and cleaned while the baking soda has an effect on the whiteness of the enamel in the dentures.

When the dentures are removed from The Solution, the dentures are brushed with a toothbrush and a toothpaste and then rinsed with cold water to remove the toothpaste. The dentures are then ready for use, without having encountered effervescence.

Efferdent™ is an example of the previously known denture cleaners that rely on effervescence for the cleaning of dentures. The directions for use printed on a container of Efferdent™ are as follows:

“1. Drop one tablet into enough very warm water (not hot) to cover denture or appliance.

2. Place denture into effervescing solution, which will change from blue to clear.

3. After 15 minutes, remove and rinse denture thoroughly. Denture is clean and odor free.

4. For best results, brush dentures using the Efferdent solution and then rinse again.” (Emphasis added)

It is the “very warm water” that causes effervescence, and the “very warm water” also causes deterioration of dentures. Presumably, the purchaser of Efferdent™ is warned to use water that is “not hot” because the effervescence increases with the temperature of the water and the increase in effervescence would increase deterioration of the denture. The manufacturer of Efferdent™, apparently knowing the damage effervescence does to the denture, and knowing that the effervescence increases with the temperature of the water, is being ‘user friendly’ in warning the purchaser of Efferdent™ to use water that is “not hot”.

The method of cleaning dentures with The Solution includes these steps:

1. Provide a bowl to hold the dentures;

2. Place the dentures in the bowl;

3. Provide a quantity of The Solution at least sufficient to cover the dentures in the bowl;

4. Pour enough of The Solution in the bowl to cover the dentures;

5. Leave the dentures submerged in The Solution at least 15 minutes, preferably overnight;

6. Then, remove the dentures from The Solution;

7. Preferably, brush the dentures with a toothbrush and toothpaste; and

8. Rinse the dentures with cold water.

The Solution is capable of sanitizing toothbrushes by simply immersing the bristles of a toothbrush in The Solution.

The Solution is also capable of functioning as a safe and effective mouthwash by placing a quantity of The Solution in the mouth and swishing it around, without swallowing, and spitting it out.

There is thus provided a method and means for controlling bacteria in the mouth by killing harmful bacteria on dentures without damage to the dentures. The scope of protection is defined in the following claims.

I claim:

1. The solution for cleaning and sanitizing dentures having a plastic gum portion and an enamel tooth portion, said solution comprising water, hydrogen peroxide, phosphoric acid, sodium lauryl sulfate, and baking soda.

2. The invention of claim 1 wherein the solution includes a desired flavoring.

3. The invention of claim 1 wherein the solution includes a desired color or colors.

4. The invention of claim 1 wherein said solution is made from a main mix and wherein the main mix comprises:
A) 90.81% water,
B) 9% of 50% hydrogen peroxide
C) 0.04% phosphoric acid
D) 0.10% sodium lauryl sulfate, and
E) 0.05% baking soda.

5. A method of sanitizing and cleaning dentures without effervescence, wherein said dentures have a plastic gum portion and an enamel tooth portion, said method comprising:
a) providing a bowl of a suitable size to immerse said dentures in a liquid solution;
b) providing a liquid solution in the bowl, said liquid solution comprising:
i) water,
ii) 50% hydrogen peroxide,
iii) sodium lauryl sulfate,
iiv) phosphoric acid, and
v) baking soda,
c) immersing the dentures in the liquid solution in the bowl for at least 15 minutes and preferably overnight while the 50% hydrogen peroxide and the potassium phosphate function to sanitize the denture, the sodium lauryl sulfate and the baking soda function to clean the denture, and the baking soda also functions to clean and whiten the enamel portion of the denture.
